(19) United States
(12) Patent Application Publication
(10) Pub. No.: US 2015/0173812 A1
(43) Pub. Date: Jun. 25, 2015

(54) HINGABLE AND FIXABLE BONE PLATE SYSTEM

(71) Applicant: Marcos V. MASSON, Houston, TX (US)

(72) Inventor: Marcos V. MASSON, Houston, TX (US)

(21) Appl. No.: 14/274,161

(22) Filed: May 9, 2014

Related U.S. Application Data
(60) Provisional application No. 61/826,731, filed on May 23, 2013.

(51) Int. Cl. A61B 17/80

(52) U.S. Cl. CPC A61B 17/80

(57) ABSTRACT

A bone plate apparatus for securing a bone or a bone fragment in a proper position with respect to another bone or a bone fragment has a plate with a first portion and a second portion in which the first portion is hingedly connected to the second portion, and a fixing element cooperative with the first and second portions so as to fix an angular orientation of the first portion with respect to the second portion. The fixing element is a screw that is rotatable in the hinge in one direction such that the first portion is freely pivotable with respect to the second portion and rotatable in an opposite direction so as to fix an angular orientation with the first portion with respect to the second portion. Each of the first portion and the second portion has holes formed therein suitable for the receipt of bone screws such that the portions can be affixed against a surface of the bone or the bone fragment.

### RELATED U.S. APPLICATIONS

The present application claims priority from U.S. Provisional Patent Application Ser. No. 61/826,731, filed on May 23, 2013 and entitled “Hingeable and Fixable Bone Plate System”.

### STATEMENT REGARDING FEDERALLY SPONSORED RESEARCH OR DEVELOPMENT

Not applicable.

### REFERENCE TO MICROFICHE APPENDIX

Not applicable.

### BACKGROUND OF THE INVENTION

1. Field of the Invention

The present invention relates to bone plates. More particularly, the present invention relates to bone plates that can be properly manipulated for application to fractured or osteotomically-separated bone. More particularly, the present invention the relates to bone plates in which one portion can be pivoted with respect to another portion and then locked in place.

2. Description of Related Art Including Information Disclosed Under 37 CFR 1.97 and 37 CFR 1.98

Orthopaedic implants have evolved into many types of devices to assist in arthrodesis and correction of bone defects of a congenital, degenerative, or trauma-related nature. Among the various types of orthopaedic implants are plate-type devices. Plate-type devices, like most devices, are temporary devices attached to stabilize two bone fragments or two bones until healing of the fragments or fusion of the two bones has occurred.

These devices are designed to be load sharing rather than load bearing. Load bearing devices typically carry all or bear all the stress. This is sometimes referred to as “stress shielding”. Load sharing transfers some amount of the stress from the device to the bone itself. This transfer of load to the bone causes stress and this stress becomes the mechanism that triggers the body to start the healing or fusion process.

Some applications require different types of fastener devices, such as screws, pins, staples, or cerclage wire, in conjunction with the plate devices to secure them to the bone to provide the required stabilization. Many fasteners are designed specifically for the two different types of bones within the body.

In certain applications where the device are used in close proximity to a joint, the device should be designed such that it does not cause damage or have adverse effects to the articular surfaces of the joint. Further considerations of implant design should also be given to ensure that ligaments and tendon structures, usually located close to the joint, that come into contact with the implant are not compromised in any way by excess material, rough surfaces, or sharp edges. The profile of the present plate constructions has been designed with these considerations.

In many osteotomically-separated bones, there is one bone fragment that is displaced with respect to another bone fragment. In order to effectively use the bone plate, the surgeon must take certain steps so as to properly align the separated bone fragments. These often require the use of certain retractors and other manipulation devices so as bring the bone fragments into proper alignment. Once the alignment is achieved, then the bone plate can be secured by screws or other members. Unfortunately, during the manipulation of the bone fragments so as to bring such bone fragments to their proper position, the bones may revert back to an offset arrangement prior to the installation of the bone plate. As such, it would be desirable to be able to utilize the bone plate for the purposes of properly aligning the separated bone fragments.

In other circumstances, a planar-type bone plate may not be suitable for the particular fracture location. In certain circumstances, such as the relationship between the humerus and the humeral head, there is desired angular relationship between such structures. If, as a result of a fracture, the humeral head should be angularly offset in an undesired relationship with respect to the humerus, the surgeon is required to make certain decisions, during the surgery, as to the properly locate of the humeral head prior of the placement of the bone plate. As such, the ultimate resulting fusion may cause the humeral head to be at an undesired angle with respect to the humerus. This can result in difficulties for the patient. As such, it would be desirable to provide a bone plate which can be properly manipulated so as to at adapt to the particular anatomy of the patient at the time that the surgical procedure is being carried out.

In the past, various patent have issued with respect to bone plates. For example, U.S. Pat. No. 5,085,660, issued on Feb. 4, 1992 to K. C. Lin, describes a locking plate system that has multiple locking pins, each with one end formed as a screw to lock in the pending fixation bones or vertebral tubercles, with another end defining a rectangular or similarly shaped locking post having a threaded locking end. There is formed a stopping protrusion near the locking post. A plate defines multiple locking bores disposed at one side to be placed over the locking post end until the plate reaches the stopping protrusion on the locking pin. The plate defines multiple threaded screwing bores near the other side to receive the locking pin screw.

U.S. Pat. No. 6,224,602, issued on May 1, 2001 to K. Hayes, provides a bone stabilization plate with a secured-locking mechanism for cervical fixation. The bone plate assembly has a fixation plate held in position in a bone by a plurality of fasteners. The fasteners extend through holes in the bone plate to engage the bone below. The assembly includes a locking plate which is secured to the fixation plate by means of a lock screw extending through an opening in the locking plate. The locking plate has a dovetailed connection to engage the flanges defining the exterior edges of a channel and the fixation plate. The locking plate has a sliding connection from a first position where it has at least partial openings corresponding to the openings of the fixation plate for the fasteners. In a second position, the edge of the plate surrounding the opening overlaps the fastener openings so as to lock these openings against the fastener.

U.S. Pat. No. 7,722,653, issued on May 25, 2010 to Young et al., discloses a locking bone plate particularly suited to tibial plateau-leveling osteotomy and a distal femoral osteotomy. The bone plate has a main longitudinal axis, a bone-contacting bottom side (not shown) and a top side with at least three sets of overlapping holes which communicate through the plate from the top to the bottom side. The sets of overlapping holes define threaded apertures having multifaceted surfaces. When applied to a bone, two sets of such overlapping holes are located so as to lie on opposite sides of an osteotomy site and on the tibial plate. A third is aligned at approximately 60° with respect to the longitudinal axis.

U.S. Pat. No. 8,246,660, issued on Aug. 21, 2012 to Boris et al., shows an implantation tool for spacing a first section of a divided lamina apart from a second section of the divided lamina in connection with a laminoplasty procedure. The apparatus is placed with an implantation tool and fastened in place by a locking plate that is held in place by a locking plate fastener driven into the tool-receiving hole in the apparatus.

U.S. Pat. No. 8,257,407, issued on Sep. 4, 2012 to H. E. Aryan, provides a bone plate system and method. This bone plate system is utilized for attaching the plate between two or more bones or bone fragments. The system includes distraction pins having a threaded bone attachment or screw section and a detachable shaft section. The screw section of the distraction pins remain in the bone and are used to affix a plate to the bones.

U.S. Pat. No. 8,287,542, issued on Oct. 16, 2012 to D. Wolter, provides a repositioning and fixation system for bone fragments. At least two bone plates are designed to be interconnected. Each bone plate is provided with a connector element for connection to another bone plate. The connector element of a first bone plate comprises a plurality of projections. The connector element of a second bone plate comprises an opening. In a first connection state, the projections engage in the opening and produce a hinge-type connection between the first bone plate and the second bone plate. In a second connection state, the projections are tensioned and produce a rigid connection between the first bone plate and the second bone plate.

U.S. Patent Publication No. 2004/0127903, published on Jul. 1, 2004 to Schlapfer et al., provides a bone fixation device that includes a pair of bone plates and a longitudinal carrier. The longitudinal carrier can be used to permit the bone plates to telescope with respect to one another, such as in a direction parallel to the longitudinal axis of the vertebral column. A joint may be used to permit swivelling of the plates with respect to each other.

U.S. Patent Publication No. 2007/0083202, published on Apr. 12, 2007 to Eli Running et al., provides an intramedullary bone plate with sheath having an intramedullary stem, a bone plate head, and a neck that connects the intramedullary stem to the bone plate head in a manner where the stem and head are offset from each other longitudinally and axially in the sagittal plane. The bone plate head includes a sheath recess wherein non-threaded bone screw holes and a threaded sheath screw hole are located. Bone screws are inserted through the sheath recess and oriented at set angles allowing for bone fragment fixation and fracture reduction.

It is an object of the present invention to provide a bone plate having a first portion that can be angularly adjusted with respect to a second portion.

It is an object of the present invention to provide a bone plate assembly that allows the angular relationship between the portions of the bone plate to be fixed.

It is still another object of the present invention to provide a bone plate assembly which allows the portions to be angularly adjusted to a predetermined angle.

It is still a further object of the present invention to provide a bone plate assembly that facilitates the ability of a surgeon to bring the bone fragments into proper alignment with each other.

It is still another object of the present invention to provide a bone plate assembly which allows the surgeon to predetermine the proper angular relationship between the bone fragments.

These and other objects and advantages of the present invention will become apparent from a reading of the attached specification and appended claims.

### BRIEF SUMMARY OF THE INVENTION

The present invention is a bone plate assembly having a first portion, a second portion, a hinge connected to the first and second portions, and a fixing means cooperative with the first and second portions so as to fix an angular orientation of the first portion with respect to the second portion. In particular, this “fixing means” includes a lock that is cooperative with the hinge so as to fix the portions in a desired angular orientation when desired. As used herein, the “fixing means” can include a variety of mechanisms. In particular, in the preferred embodiment, the “fixing means” is a screw that extends through the hinge. A rotation of the screw will create a friction between the hinging elements so as to fix the first portion with respect to the second portion. Alternatively, a cam-type of locking mechanism can be provided with in the hinge.

In the bone plate assembly of the present invention, the first portion has a bone-contacting surface. A plurality of holes are formed through the first portion. The second portion is an elongated panel. This elongated panel also has a bone-contacting surface. Holes are formed through the elongated panel of the second portion.

The hinge is connected to the first and second portions. The hinge allows one portion to pivot with respect to the second portion. The fixing means can include a screw that is engaged with the hinge. A rotation of the screw in one direction causes the connection between first and second portions to be released such that the first portion freely pivots with respect to the second portion. A rotation of the screw in the opposite direction serves to fix the hinge such that the angular orientation between the first and second portions is fixed.

When the bone plate assembly is applied to bone fragments, such as the humeral head and the humerus, the first portion could be attached to one bone fragment and the second portion can be connected to another bone fragment. Since the angular orientation between the first and second portions is fixed, the surgeon can apply a leveraging force upon the second portion so as to draw the bone fragment attached to the first portion upwardly to a desired angle. Once the second portion resides against the other bone fragment, the screws can be inserted through the holes of the second portion so as to secure the second portion. In this arrangement, the bone fragment that is secured to the first portion will be at its desired angular orientation with respect to the second fragment.

### BRIEF DESCRIPTION OF THE SEVERAL VIEWS OF THE DRAWINGS

FIG. 1 is a plan view of the bone plate assembly in accordance with preferred embodiment of the present invention.

FIG. 2 is a side elevational view of the bone plate assembly shown in a linear orientation.

FIG. 3 is a side elevational view of the bone plate assembly of the present invention showing the portions in an angular orientation.

FIG. 4 shows an initial step in the application of the bone plate assembly of the present invention to a fractured humerus.

FIG. 5 shows a later step in the use of the bone plate assembly of the present invention in association with the fractured humerus.

### DETAILED DESCRIPTION OF THE INVENTION

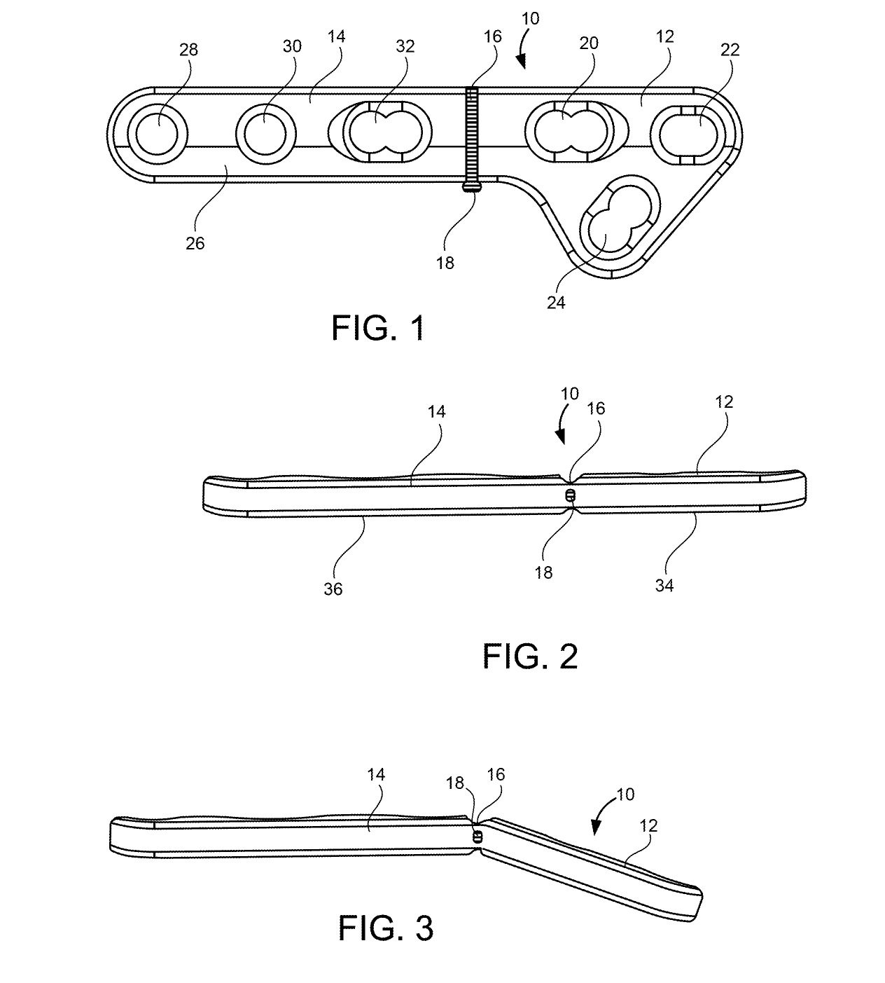

Referring to FIG. 1, there is shown the bone plate assembly 10 in accordance with the teachings of the present invention. The bone plate assembly 10 includes a first portion 12 and a second portion 14. A hinge 16 joins the first portion 12 to the second portion 14. A lock 18 is cooperative with the hinge 16 and cooperative with the first portion 12 and the second portion 14 so as to fix or release an angular orientation between the first portion 12 and the second portion 14.

In FIG. 1, it can be seen that the first portion 12 has a generally triangular configuration. However, the configuration of the first portion 12 can take on a wide variety of shapes depending, in particular, on the desired application of the bone plate assembly 10. The first portion 12 includes holes 20, 22 and 24 that are formed therethrough. Holes 20, 22 and 24 are suitable for the receipt of bone screws therein. Through the use of the bone screws, as extending through the holes 20, 22 and 24, the first portion 12 can be secured to a bone fragment in a strong manner. The concept of the present invention contemplates a wide variety of hole configurations and shapes. As such, this particular configuration of the shape of the first portion 12 or the arrangement of the holes 20, 22 and 24 should not be limiting of the present invention.

The second portion 14 is in the form of an elongated panel 26. The elongated panel 26 has one end connected to the hinge 16. A plurality of holes 28, 30 and 32 are formed through the thickness of the elongated panel 26. Holes 28, 30 and 32 are suitable for receipt of bone screws therein. In the concept of the present invention, the second portion 14 of the bone plate assembly 10 is contemplated to be attached to another bone fragment. Suitable bone screws, as extending through the holes 28, 30 and 32 strongly affix the elongated panel 26 to the surface of the bone.

The hinge 16 is a conventional hinge that allows a pivotal relationship between the first portion 12 and the second portion 14. The lock 18 is a “fixing means” that is cooperative with the hinge 16 so as to allow the surgeon to fix the desired angular orientation between the first portion 12 and the second portion 14. As shown in the FIG. 1, the lock 18 is in the nature of screw that is engaged with the hinge so as to exert strong frictional forces upon the hinge so as to effectively lock the first portion 12 with respect to the second portion 14. In particular, a rotation of the screw in one direction causes the connection to be released such that the first portion 12 moves with respect to the second portion 14. A rotation of the screw 18 in the opposite direction fixes the hinge 16 such that the angular orientation between the first portion 12 and the second portion 14 is fixed.

FIG. 2 illustrates the bone plate assembly 10 of the present invention in a linear orientation. As can be seen, the first portion 12 extends in planar relationship to the second portion 14. The first portion 12 is fastened by a hinge 16 with second portion 14. The lock 18 is illustrated as engaged with the hinge 16. As such, the lock 18 can be suitably rotated in one direction so that the first portion 12 is movable with respect to the second portion 14 and rotated in an opposite direction such that the first portion 12 is fixed with respect to second portion 14.

The first portion 12 includes a bone-contacting surface 34 on one side thereof. The second portion 14 also includes a bone containing portion 36 on one side thereof. Within the concept of the present invention, if it is desired to maintain a linear orientation between the bone fragments, then the bone plate assembly 10, as shown in FIG. 2, can be utilized. The bone-contacting surfaces 34 and 36 can be placed against the bones so that the bones assume a proper linear alignment.

In FIG. 3, it can be seen that the first portion 12 is angularly offset with respect to the second portion 14. In particular, the first portion 12 extends at an obtuse angle with respect to the second portion 14. In these circumstances, the lock 18 is released from the configuration shown in FIG. 2 so that the surgeon can pivot the first portion 12 with respect to the second portion 14. The lock 18 can then be rotated so as to fix the hinge 16 so that the first portion 12 resides in a strongly secured angular orientation with respect to the second portion 14.

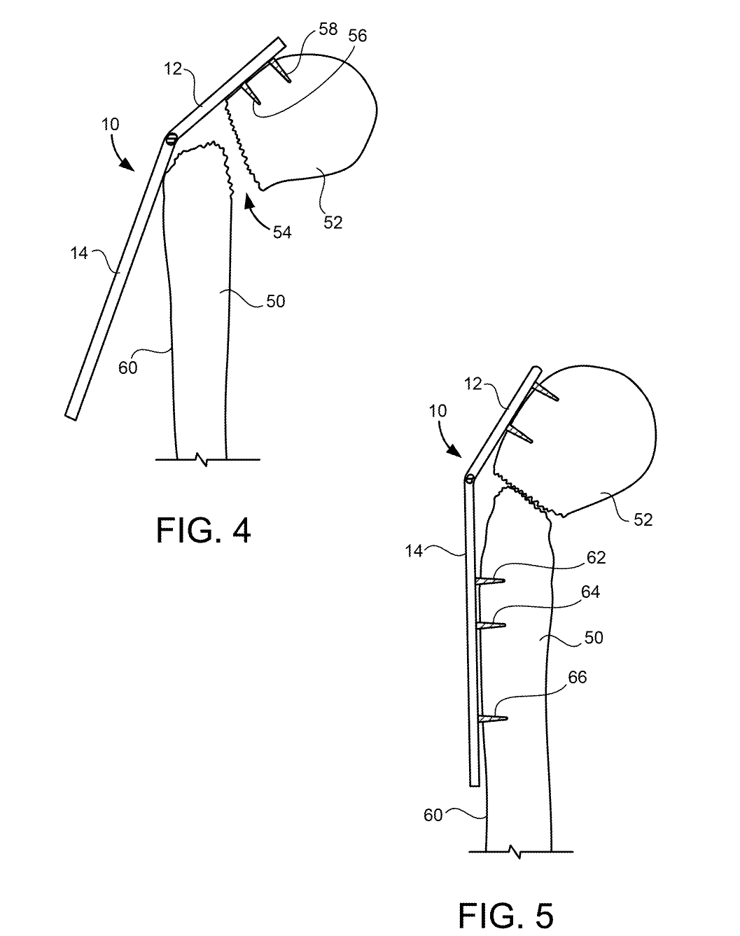

FIG. 4 shows the use of the bone plate assembly 10 of the present invention in association with bone fragments. In particular, in FIG. 4, the “bone fragments” are in the nature of the humerus 50 and a humeral head 52. The fracture 54 is illustrated as separated between the humerus 50 and the humeral head 52.

When a fracture 54 occurs, the humeral head 52 may move to an undesirable orientation with respect to the humerus 50. Under certain circumstances, the humeral head 52 will sink downwardly in the shoulder. In the past, the surgeon would have to use certain manipulating so as to move the humeral head 52 back to its desired orientation with respect to the humerus 50. This can be traumatic for the patient, consume additional time of the surgeon, and result in a potentially inaccurate placement of the humeral head 52 with respect to the humerus 50. The bone plate assembly 10 is intended to overcome such a problem.

In FIG. 4, it can be seen that the first portion 12 of the bone plate assembly 10 is fixed at an obtuse angle with respect to the second portion 14. The second portion 14 is secured to the humeral head 52 through the use of the bone screws 56 and 58. As such, the first portion 12 is securely fastened to the humeral head 52. The second portion 14 extends outwardly of the outer surface 60 of the humerus 50 by a certain distance. The upper portion of the humerus 50 can serve as a fulcrum for the bone plate assembly 10 so as to allow the bone plate assembly 10 to properly move the humeral head 52 into a desired orientation with respect to the humerus 50. In order to fix the proper angular orientation of the first portion 12 with respect to the second portion 14, suitable imaging can be carried out by the surgeon, or records inspected so as to provide information as the desired angular orientation between the humeral head 52 and the humerus 50.

FIG. 5 shows the placement of the humeral head 52 back onto the upper end of the humerus 50. The surgeon will move the second portion 42 of the bone plate assembly 10 downwardly so as to reside against the outer surface 60 of the humerus 50. Suitable bone screws 62, 64 and 66 can be inserted so as to fixedly attach the second portion 14 to the humerus 50. Because of the strong leveraging forces resulting from the angularly outwardly extending orientation of the second portion 14 (as shown in FIG. 4), the movement of the second portion 14 back to the outer surface 60 of the humerus 50 will cause the humeral head 52 to concomitantly move back to its desired position. In other words, the first portion 12 will draw the humeral head 52 back upwardly at an angle to the positioning in which it resides against the upper end of the humerus 50. The bone plate assembly 10 can then remain in place until proper fusion between the bone fragments occurs.

The foregoing disclosure and description of the invention is illustrative and explanatory thereof. Various changes in the details of the illustrated construction or step in the described method can be made within the scope of the present claims without departing from the true spirit of the invention. The present invention should only be limited by the following claims and their legal equivalents.

I claim:

1. An apparatus comprising:
a plate having a first portion and a second portion, said first portion being connected by a hinge to said second portion; and
a fixing means cooperative with said first portion and said second portion so as to fix an angular orientation of said first portion with respect to said second portion for fixing a position of a pair of bones or bone fragments.

2. The apparatus of claim 1, said first portion having a bone-contacting surface thereon, said first portion having at least one hole formed therethrough.

3. The apparatus of claim 1, said second portion being an elongated panel having a bone-contacting surface, said second portion having at least one hole formed therethrough.

4. The apparatus of claim 1, said fixing means comprising a screw that is engaged with said hinge.

5. The apparatus of claim 4, said screw being rotatable in said hinge in one direction such that said first portion is freely pivotable with respect to said second portion and rotatable in an opposite direction so as to fix an angular orientation of said first portion with respect to said second portion.

6. The apparatus of claim 1, further comprising:
a first screw extending through a hole formed in said first portion, said first screw suitable for securing into the bone or the bone fragment.

7. The apparatus of claim 6, further comprising:
a second screw extending through a hole formed in said second portion, said second screw suitable for engaging with another bone or another bone fragment.

8. The apparatus of claim 1, said first portion having a planar bone-contacting surface, said second portion having another planar bone-contacting surface.

9. The apparatus of claim, said first and second screws formed of an osteconductive material.

10. A surgical process for affixing the bone plate to a first bone into a second bone, the bone plate having a first portion pivotally connected by a hinge to a second portion, the surgical process comprising:
affixing the first portion to the first bone;
fixing an angular orientation of said first portion with respect to said second portion;
leveraging the second portion toward the second bone so as to cause the first bone to move in a desired orientation with respect to the second bone; and
affixing the second portion to the second bone.

11. The surgical process of claim 10, at least one of the first bone and the second bone being a bone fragment.

12. The surgical process of claim 11, the step of affixing the first portion comprising:
inserting at least one screw through a hole in the first portion; and
screwing the screw into the first bone until a bone-contacting surface of the first portion resides against the first bone.

13. The surgical process of claim 11, the step of fixing an angular orientation comprising:
tightening a screw into the hinge until the first portion is fixed into a first angular orientation with respect to the second portion.

14. The surgical process of claim 11, the step of fixing an angular orientation comprising:
locking the first portion in a desired angular orientation with respect to the second portion.

15. The surgical process of claim 11, the step of leveraging comprising:
applying a force toward the second portion in a direction toward the second bone;
moving the second portion toward the second bone until a bone-contacting surface of the second portion contacts the second bone.

16. An apparatus comprising:
a plate having a first portion and a second portion, said first portion being connected by a hinge to said second portion; and
a friction fastener positioned in said hinge, said friction fastener being rotatable in a direction so as to lock an angular orientation with said first portion with respect to said second portion.

17. The apparatus of claim 16, said friction fastener being a screw connected within said hinge.

18. The apparatus of claim 16, further comprising:
a first screw extending through a hole formed in said first portion, said first screw suitable for engaging a bone or a bone fragment; and
a second screw extending through a hole formed in said second portion, said second screw suitable for securing against another bone or another bone fragment.

19. The apparatus of claim 16, said first portion having a planar bone-contacting surface, said second portion having another planar bone-contacting surface.

20. The apparatus of claim 16, said first portion having a bone-contacting surface thereon, said first portion having at least one hole formed therethrough, said second portion being an elongated panel having a bone-contacting surface, said second portion having at least one hole formed therethrough.
